(19) United States
(12) Patent Application Publication
(10) Pub. No.: US 2012/0010195 A1
(43) Pub. Date: Jan. 12, 2012

(54) IMIDAZOLE DERIVATIVES

(75) Inventors: Raphael Dumeunier, Stein (CH); Clemens Lamberth, Stein (CH); Stephan Trah, Stein (CH)

(73) Assignee: SYNGENTA CROP PROTECTION, LLC, Greensboro, NC (US)

(21) Appl. No.: 13/256,074

(22) PCT Filed: Feb. 8, 2010
(86) PCT No.: PCT/EP2010/051476

§ 371 Date: Sep. 12, 2011

(30) Foreign Application Priority Data
Mar. 12, 2009 (GB) 0904315.9

(51) Int. Cl. A01N 43/46; A01P 3/00; C07D 403/04

(52) U.S. Cl. 514/217.09; 540/603

(57) ABSTRACT

The present invention relates to novel imidazole derivatives of formula (I) having microbiocidal, in particular fungicidal, activity, to processes for their preparation and intermediates used in their preparation, to agrochemical compositions comprising them and to the use in agriculture or horticulture for controlling or preventing infestation of plants, harvested food crops, seeds or non-living materials by phytopathogenic microorganisms, preferably fungi.

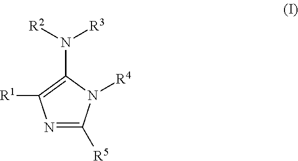

The present invention relates to novel imidazole derivatives having microbiocidal, in particular fungicidal, activity, to processes for their preparation and intermediates used in their preparation, to agrochemical compositions comprising them and to the use in agriculture or horticulture for controlling or preventing infestation of plants, harvested food crops, seeds or non-living materials by phytopathogenic microorganisms, preferably fungi.

In addition, the present invention also relates to the use of these novel imidazole derivatives as plant growth regulators (PGRs).

Furthermore, the present invention also relates to compositions comprising the novel imidazole derivatives that improve plants, a process which is commonly and hereinafter referred to as “plant health”.

The present invention further relates to the use of these novel imidazole derivatives in the treatment of cancer and to pharmaceutical compositions comprising at least one of these compounds as active component.

Accordingly the present invention provides a compound of formula I:

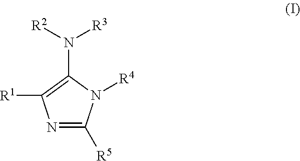

wherein
R¹ is halogen or C₁-C₆alkyl;
R² is an optionally substituted C₁-C₈alkyl, C₂-C₆alkenyl, C₂-C₆alkynyl, C₁-C₇haloalkyl, C₃-C₉cycloalkyl, C₁-C₁₁alkylcycloalkyl or C₁-C₈heterocyclyl;
R³ is hydrogen or C₁-C₇alkyl; or
R² and R³ together with the nitrogen atom to which they are attached form an optionally substituted heterocylic ring;
R⁴ is an optionally substituted aryl or heteroaryl; and
R⁵ is halogen;
said optional substituents comprising one or more substituents independently selected from halogen, alkyl, haloalkyl, cycloalkyl, cycloalkylalkyl, alkenyl, haloalkenyl, cycloalkenyl, alkynyl, haloalkynyl, alkyloxy, haloalkyloxy, cycloalkoxy, alkenyloxy, haloalkenyloxy, alkynyloxy, haloalkenyloxy, alkylthio, haloalkylthio, cycloalkylthio, alkenylthio, alkynylthio, alkylcarbonyl, haloalkylcarbonyl, cycloalkylcarbonyl, alkenylcarbonyl, alkynylcarbonyl, alkoxyalkyl, cyano, nitro, hydroxy, mercapto, amino, alkylamino and dialkylamino;
or an agrochemically usable salt form thereof.

Halogen means fluorine, chlorine, bromine and iodine.

Alkyl, alkenyl or alkynyl radicals may be straight-chain or branched.

Unless otherwise indicated, alkyl, on its own or as part of another substituent, may contain from 1 to 12 carbon atoms, preferably 1 to 8, more preferably 1 to 6. Examples of alkyl include methyl, ethyl, propyl, butyl, pentyl, hexyl and the isomers thereof, for example, isopropyl, isobutyl, sec-butyl, tert-butyl, isopentyl and tert-pentyl.

Unless otherwise indicated, alkenyl, on its own or as part of another substituent, may contain from 2 to 12 carbon atoms, preferably 2 to 8, more preferably 2 to 6. Examples of alkenyl include ethenyl, allyl, 1-propenyl, buten-2-yl, buten-3-yl, penten-1-yl, penten-3-yl, hexen-1-yl and 4-methyl-3-pentenyl.

Unless otherwise indicated, alkynyl, on its own or as part of another substituent, may contain from 2 to 12 carbon atoms, preferably 2 to 8, more preferably 2 to 6. Examples of alkynyl include ethynyl, propyn-1-yl, propyn-2-yl, butyn-1-yl, butyn-2-yl, 1-methyl-2-butynyl, hexyn-1-yl or 1-ethyl-2-butynyl.

Haloalkyl groups may contain one or more identical or different halogen atoms, and includes, for example, CH₂Cl, CHCl₂, CCl₃, CH₂F, CHF₂, CF₃, CF₃CH₂, CH₃CF₂, CF₃CF₂ and CCl₃CCl₂.

Unless otherwise indicated, cycloalkyl, on its own or as part of another substituent, may contain from 3 to 12 carbon atoms, preferably 4 to 8, more preferably 5 to 6. Examples of cycloalkyl include cyclopropyl, cyclobutyl, cyclopentyl and cyclohexyl.

“Heterocyclyl”, as used herein refers to a saturated or partially unsaturated cyclic hydrocarbon containing from 3 to 10 ring-atoms up to 4 of are hetero-atoms selected from nitrogen, oxygen and sulfur, and may be optionally substituted by one or more groups independently selected from halogen, nitro, cyano, alkyl, alkoxy. Examples of heterocyclyl groups are oxiranyl, azetidinyl, tetrahydrofuranyl, thiolanyl, pyrrolidinyl, pyrrolinyl, imidazolidinyl, imidazolinyl, sulfolanyl, dioxolanyl, dihydropyranyl, tetrahydropyranyl, piperidinyl, pyrazolinyl, pyrazolidinyl, dioxanyl, morpholinyl, dithianyl, thiomorpholinyl, piperazinyl, azepinyl, oxazepinyl, thiazepinyl, thiazolinyl and diazapanyl.

Aryl includes phenyl, naphthyl, anthracenyl, phenanthrenyl and biphenyl, with phenyl being preferred.

Heteroaryl means a cyclic, aromatic hydrocarbon comprising mono-, bi- or tricyclic systems, containing 3 to 14, preferably 5 to 10, more preferably 6 to 8, ring-atoms, including 1 to 6, preferably 1 to 4, heteroatoms independently selected from nitrogen, oxygen and sulfur. Examples are furyl, thienyl, pyrrolyl, imidazolyl, pyrazolyl, thiazolyl, isothiazolyl, oxazolyl, isoxazolyl, oxadiazolyl, thiadiazolyl, triazolyl, tetrazolyl, pyridyl, pyridazinyl, pyrimidinyl, pyrazinyl, triazinyl, tetrazinyl, indolyl, benzothiophenyl, benzofuranyl, benzimidazolyl, indazolyl, benzotriazolyl, benzothiazolyl, benzoxazolyl, quinolyl, isoquinolyl, phthalazinyl, quinoxalinyl, quinazolinyl, cinnolinyl and naphthyridinyl.

Typical examples for optionally substituted aryl include 2,4-difluorophenyl, 2,5-difluorophenyl, 2,6-difluorophenyl, 3,4-difluorophenyl, 2,4-dichlorophenyl, 2,5-dichlorophenyl, 2,6-dichlorophenyl, 3,4-dichlorophenyl, 3,4-dimethylphenyl, 3,4-dimethoxyphenyl, 2-chloro-4-fluorophenyl, 2-chloro-5-fluorophenyl, 2-chloro-6-fluorophenyl, 3-chloro-4-fluorophenyl, 3-chloro-6-fluorophenyl, 3-chloro-4-methylphenyl, 3-chloro-4-methoxyphenyl, 4-chloro-2-fluorophenyl, 4-chloro-3-fluorophenyl, 4-chloro-3-methylphenyl, 4-chloro-3-methoxyphenyl, 3-fluoro-4-methoxyphenyl, 3-fluoro-4-methylphenyl, 4-fluoro-3-methoxyphenyl, 4-fluoro-3-methylphenyl, 3-methoxy-4-methylphenyl, 4-methoxy-3-methylphenyl, 2,6-difluoro-4-methylphenyl, 2,6-difluoro-4-trifluoromethylphenyl, 2,6-difluoro-4-methoxyphenyl, 2,6-difluoro-4-trifluoromethoxyphenyl, 2,6-difluoro-4-cyanophenyl, 2,4,6-trifluorophenyl and 2,5,6-trifluorophenyl.

Typical examples for optionally substituted heteroaryl include 3,5-dichloropyridin-2-yl, 3,5-difluoropyridin-2-yl, 3-chloro-5-fluoropyridin-2-yl, 3-chloro-5-methylpyridin-2-yl, 3-chloro-5-trifluoromethylpyridin-2-yl, 3-chloro-5-methoxypyridin-2-yl, 3-chloro-5-trifluoromethoxypyridin-2-yl, 3-chloro-5-cyanopyridin-2-yl, 5-chloro-3-fluoropyridin-2-yl, 3-fluoro-5-methylpyridin-2-yl, 3-fluoro-5-trifluoromethylpyridin-2-yl, 3-fluoro-5-methoxypyridin-2-yl, 3-fluoro-5-trifluoromethoxypyridin-2-yl and 3-fluoro-5-cyanopyridin-2-yl.

The presence of one or more possible asymmetric carbon atoms in a compound of formula I means that the compounds may occur in optically isomeric, that means enantiomeric or diastereomeric forms. As a result of the presence of a possible aliphatic C═C double bond, geometric isomerism, that means cis-trans or (E)-(Z) isomerism may also occur. Also atropisomers may occur as a result of restricted rotation about a single bond. Formula I is intended to include all those possible isomeric forms and mixtures thereof. The present invention intends to include all those possible isomeric forms and mixtures thereof for a compound of formula I.

In each case, the compounds of formula I according to the invention are in free form or in an agronomically usable salt form.

In a first embodiment, compounds of formula I according to the invention have R¹ which is fluoro, chloro, bromo, iodo or CᵣC₅alkyl.

In a second embodiment, compounds of formula I according to the invention have R² which is an optionally substituted C₁-C₇alkyl, C₂-C₅alkenyl, C₂-C₅alkynyl, C₁-C₆haloalkyl, C₃-C₈cycloalkyl, C₁-C₁₀alkylcycloalkyl or C₁-C₇heterocyclyl.

In a third embodiment, compounds of formula I according to the invention have R³ which is hydrogen or C₁-C₆alkyl.

In a fourth embodiment, compounds of formula I according to the invention have R² and R³ together with the nitrogen atom to which they are attached, which form an optionally substituted mono or bicylic three- to ten-membered heterocyclic ring, wherein one or two ring carbon atoms are replaced by oxygen, sulphur or nitrogen

In a fifth embodiment, compounds of formula I according to the invention have R⁴ which is an optionally substituted phenyl, naphthyl, thienyl, pyridyl, quinolyl or isoquinolyl.

In a sixth embodiment, compounds of formula I according to the invention have R⁵ which is fluoro, chloro, bromo or iodo.

Preferred subgroups of compounds of formula I according to the invention are those wherein

R¹ is fluoro, chloro, bromo or C₁-C₄alkyl;
R² is an optionally substituted C₁-C₆alkyl, C₁-C₅haloalkyl, C₃-C₇cycloalkyl or C₁-C₉alkylcycloalkyl;
R³ is hydrogen or C₁-C₅alkyl; or
R² and R³ together with the nitrogen atom to which they are attached form an optionally substituted monocyclic three- to nine-membered heterocyclic ring, wherein one or two ring carbon atoms are replaced by oxygen or sulphur;
R⁴ is an optionally substituted phenyl, naphthyl, thienyl or pyridyl; and
R⁵ is fluoro, chloro or bromo.

Most preferred subgroups of compounds of formula I according to the invention are those wherein

R¹ is fluoro, chloro, bromo, or C₁-C₃alkyl;
R² is an optionally substituted ethyl, iso-propyl, sec-butyl, iso-butyl, 1,2-dimethylpropyl, 1,2,2-trimethylpropyl, 2,2,2-trifluoroethyl, 2-(1,1,1-trifluoropropyl), 2-(1,1,1-trifluorobutyl), cyclopentyl or cyclohexyl;
R³ is hydrogen or C₁-C₄alkyl; or R² and R³ together with the nitrogen atom to which they are attached form an optionally substituted mono three- to seven-membered heterocylic ring, wherein one ring carbon atoms are replaced by oxygen or sulphur;
R⁴ is 2,4,6-trifluorophenyl, 2,6-difluoro-4-methoxyphenyl or 3,5-dichloro-pyridin-2-yl; and
R⁵ is fluoro or chloro.

Especially preferred subgroups of compounds of formula I according to the invention are those wherein

R¹ is chloro or methyl;
R² is iso-butyl or 2-(1,1,1-trifluoropropyl);
R³ is hydrogen; or
R² and R³ together with the nitrogen atom to which they are attached form a 4-methyl piperidine ring;
R⁴ is 2,4,6-trifluorophenyl or 2,6-difluoro-4-methoxyphenyl; and
R⁵ is fluoro or chloro.

Preferred individual compounds are:
1-[2,5-Dichloro-3-(2,4,6-trifluoro-phenyl)-3H-imidazol-4-yl]-4-methyl-piperidine;
1-[2-Chloro-3-(2,6-difluoro-4-methoxy-phenyl)-5-methyl-3H-imidazol-4-yl]-4-methyl-piperidine;
1-[5-Chloro-2-fluoro-3-(2,4,6-trifluoro-phenyl)-3H-imidazol-4-yl]-4-methyl-piperidine;
1-[2-Chloro-5-methyl-3-(2,4,6-trifluoro-phenyl)-3H-imidazol-4-yl]-4-methyl-piperidine;
1-[2,5-Dichloro-3-(2,6-difluoro-4-methoxy-phenyl)-3H-imidazol-4-yl]-4-methyl-piperidine;
[2-Chloro-3-(2,6-difluoro-4-methoxy-phenyl)-5-methyl-3H-imidazol-4-yl]-(2,2,2-trifluoro-1-methyl-ethyl)-amine;
[2-Chloro-3-(2,6-difluoro-4-methoxy-phenyl)-5-methyl-3H-imidazol-4-yl]-isobutyl-amine;
1-[2-Chloro-5-methyl-3-(2,4,6-trifluoro-phenyl)-3H-imidazol-4-yl]-4-methyl-piperidine;
1-[2-Chloro-3-(2,6-difluoro-4-methoxy-phenyl)-5-methyl-3H-imidazol-4-yl]-4-methyl-piperidine;
[2-Chloro-5-methyl-3-(2,4,6-trifluoro-phenyl)-3H-imidazol-4-yl]-(2,2,2-trifluoro-1-methyl-ethyl)-amine;
[2-Chloro-3-(2,6-difluoro-4-methoxy-phenyl)-5-methyl-3H-imidazol-4-yl]-(2,2,2-trifluoro-1-methyl-ethyl)-amine;
sec-Butyl-[2-chloro-5-methyl-3-(2,4,6-trifluoro-phenyl)-3H-imidazol-4-yl]-amine;
sec-Butyl-[2-chloro-3-(2,6-difluoro-4-methoxy-phenyl)-5-methyl-3H-imidazol-4-yl]-amine;
[2-Chloro-5-methyl-3-(2,4,6-trifluoro-phenyl)-3H-imidazol-4-yl]-isobutyl-amine;
[2-Chloro-3-(2,6-difluoro-4-methoxy-phenyl)-5-methyl-3H-imidazol-4-yl]-isobutyl-amine;
[2-Chloro-5-methyl-3-(2,4,6-trifluoro-phenyl)-3H-imidazol-4-yl]-(1,2-dimethyl-propyl)-amine;
[2-Chloro-3-(2,6-difluoro-4-methoxy-phenyl)-5-methyl-3H-imidazol-4-yl]-(1,2-dimethyl-propyl)-amine;
4-[2-Chloro-5-methyl-3-(2,4,6-trifluoro-phenyl)-3H-imidazol-4-yl]-morpholine; and
4-[2-Chloro-3-(2,6-difluoro-4-methoxy-phenyl)-5-methyl-3H-imidazol-4-yl]-morpholine.

Compounds of Formula (I.1),

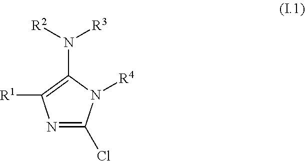

in which R¹, R², R³ and R⁴ have the meanings given above, are examples of compounds of general formula (I) and can be made as shown in the following schemes.

The compounds of formula I.1, wherein R², R³ and R⁴ are as defined for compound of formula I, and R¹ is halogen or C₁-C₄alkyl, can be obtained by reaction of a compound of formula II, wherein R², R³ and R⁴ are as defined for compound of formula I, and R¹ is halogen or C₁-C₄alkyl, with N-chlorosuccinimide (NCS) or molecular chlorine.

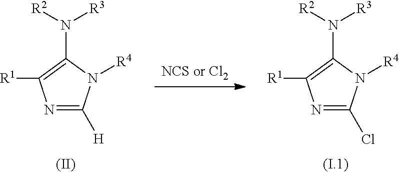

The compounds of formula I, wherein R², R³, R⁴ and R⁵ have the meanings given above, and R¹ is halogen, preferably chlorine or bromine, can be obtained by reaction of a compound of formula III, wherein R², R³ and R⁴ are as defined for compound of formula I, with at least 2 equivalents of N-chlorosuccinimide (NCS), N-bromosuccinimide (NBS) or N-iodosuccinimide (NIS).

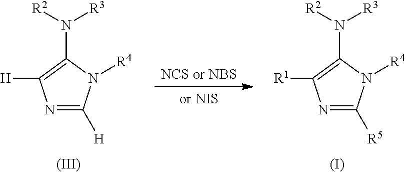

The compounds of formula II, wherein R², R³ and R⁴ are as defined for compound of formula I, and R¹ is halogen, preferably chlorine or bromine or C₁-C₄alkyl, preferably methyl, can be obtained by reaction of a compound of formula IV, wherein R², R³ and R⁴ are as defined for compound of formula I, and R¹ is halogen, preferably chlorine or bromine or C₁-C₄alkyl, preferably methyl, preferably chlorine or bromine, with sodium/mercury amalgam, in an protic solvent such as methanol, in the presence of NaH₂PO₄ or Na₂HPO₄.

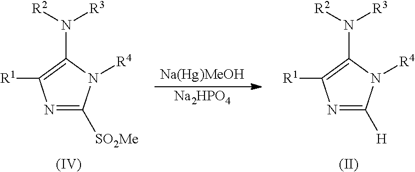

The compounds of formula IV, wherein R², R³ and R⁴ are as defined for compound of formula I, and R¹ is halogen, preferably chlorine or bromine, or C₁-C₄alkyl, preferably methyl, can be obtained by reaction of a compound of formula V, wherein R², R³ and R⁴ are as defined for compound of formula I, and R¹ is halogen, preferably chlorine or bromine, or C₁-C₄alkyl, preferably methyl, with a peracid such as meta-chloroperbenzoic acid (mCPBA).

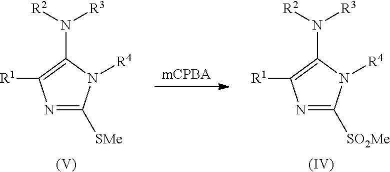

Alternatively, the compounds of formula II, wherein R², R³ and R⁴ are as defined for compound of formula I, and R¹ is hydrogen, halogen, preferably chlorine or bromine, or C₁-C₄alkyl, preferably methyl, can be obtained by reaction of a compound of formula V, wherein R², R³ and R⁴ are as defined for compound of formula I, and R¹ is hydrogen, halogen, preferably chlorine or bromine, or C₁-C₄alkyl, preferably methyl, with Raney Nickel, in an protic solvent such as ethanol.

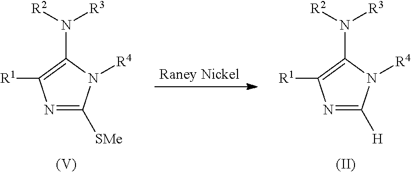

The compounds of formula V, wherein R², R³ and R⁴ are as defined for compound of formula I, and R¹ is halogen, preferably chlorine or bromine, can be obtained by reaction of a compound of formula VI, wherein R², R³ and R⁴ are as defined for compound of formula I, with N-chlorosuccinimide (NCS) or N-bromosuccinimide (NBS).

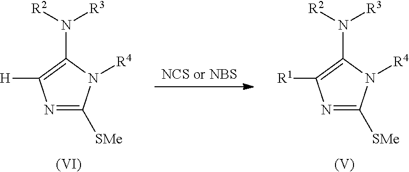

The compounds of formula V, wherein R² and R⁴ are as defined for compound of formula I, R³ is a C₁-C₆alkyl group, or R² and R³ together with the interjacent nitrogen atom is an optionally substituted heterocyclic ring, and R¹ is hydrogen or C₁-C₄alkyl, preferably methyl, can be obtained by reaction of a compound of formula VII, wherein R² and R⁴ are as defined for compound of formula I, R³ is a C₁-C₆alkyl group, or R² and R³ together with the interjacent nitrogen atom is an optionally substituted heterocyclic ring, and R¹ is hydrogen or C₁-C₄alkyl, preferably methyl, with 2,4-bis(4-methoxyphenyl)-1,3,2,4-dithiadiphosphetane 2,4-disulfide, known also under the name of Lawesson's Reagent.

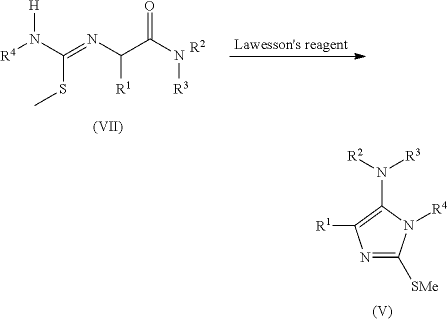

The compounds of formula VIII, wherein R² and R⁴ are as defined for compound of formula I, R⁵ is alkylthio, sulfone, hydrogen or halogen, and R¹ is hydrogen, halogen or C₁-C₄alkyl, preferably methyl, can be obtained by deprotection of a compound of formula I, wherein R² and R⁴ are as defined for compound of formula I, R⁵ is alkylthio, sulfone, hydrogen or halogen, R¹ is hydrogen, halogen or C₁-C₄alkyl, preferably methyl, and R³ is a suitable protecting group.

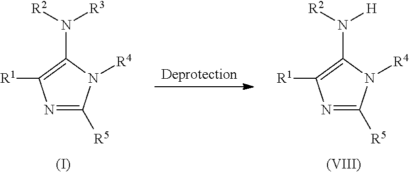

The compounds of formula VII wherein R² and R⁴ are as defined for compound of formula I, R¹ is hydrogen or C₁-C₄alkyl, preferably methyl, and R³ is C₁-C₆alkyl or a suitable protecting group, or R² and R³ together with the interjacent nitrogen atom is an optionally substituted heterocyclic ring, can be obtained by reaction of a compound of formula IX, wherein R¹, R², R³ and R⁴ are as defined for compound of formula VII, or R² and R³ together with the interjacent nitrogen atom is an optionally substituted heterocyclic ring, with methyl iodide in the presence of a base.

The compounds of formula IX, wherein R¹, R², R³ and R⁴ are as defined for compound of formula VII, or R² and R³ together with the interjacent nitrogen atom is an optionally substituted heterocyclic ring, can be obtained by reaction of a compound of formula X, wherein R¹, R² and R³ are as defined for compound of formula VII, or R² and R³ together with the interjacent nitrogen atom is an optionally substituted heterocyclic ring, with an isothiocyanate of formula XI, wherein R⁴ is as defined for compound of formula I, in an appropriate solvent such as toluene.

The compounds of formula X, wherein R¹, R² and R³ are as defined for compound of formula VII, or R² and R³ together with the interjacent nitrogen atom is an optionally substituted heterocyclic ring, can be obtained by deprotection of a compound of formula XII, wherein R¹, R² and R³ are as defined for compound of formula VII, or R² and R³ together with the interjacent nitrogen atom is an optionally substituted heterocyclic ring, in the presence of hydrochloric acid in dioxane.

The compounds of formula XIII, wherein R¹ is as defined for compound of formula VII, can be obtained by reaction with a compound of formula XIV wherein R² and R³ are as defined for compound of formula VII, or R² and R³ together with the interjacent nitrogen atom is an optionally substituted heterocyclic ring, in the presence of a coupling agent such as dicyclocarbodiimide.

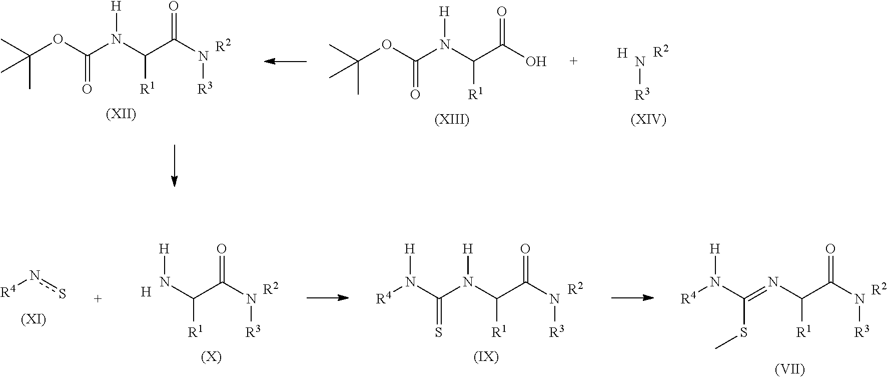

The compounds of formula XV, wherein R² and R³ are as defined for compound of formula I, or R² and R³ together with the interjacent nitrogen atom is an optionally substituted heterocyclic ring, R⁵ is alkylthio, R⁶ is C₁-C₄alkyl and R¹ is hydrogen, halogen or C₁-C₄alkyl, preferably methyl, can be obtained by reaction with a compound of formula XVI wherein R² and R³ are as defined for compound of formula I, or R² and R³ together with the interjacent nitrogen atom is an optionally substituted heterocyclic ring, R⁵ is alkylthio and R¹ is hydrogen, halogen or C₁-C₄alkyl, preferably methyl, with an alkoxide of formula LiOR⁶, NaOR⁶, KOR⁶, R⁶ being C₁-C₄alkyl.

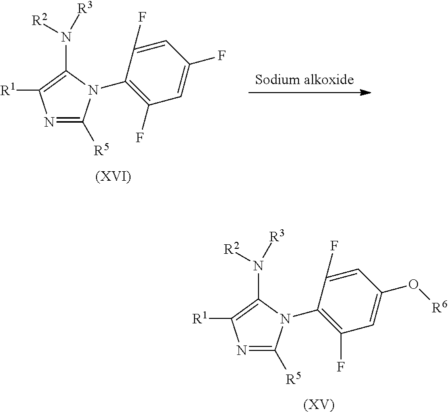

The compounds of formula XVII, wherein R², R³ and R⁴ are as defined for compound of formula I, and R⁵ is alkylthio can be obtained by reaction of a compound of formula XVI, wherein R², R³ and R⁴ are as defined for compound of formula I, and R⁵ is alkylthio, with trimethylboroxine in the presence of a transition metal catalyst.

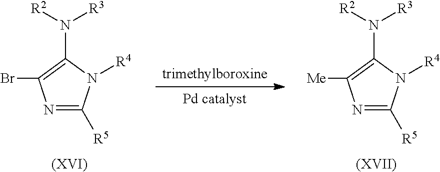

Surprisingly, it has now been found that the novel compounds of formula I have, for practical purposes, a very advantageous level of biological activity for protecting plants against diseases that are caused by fungi as well as by bacteria and viruses.

The compounds of formula I can be used in the agricultural sector and related fields of use as active ingredients for controlling plant pests or on non-living materials for control of spoilage microorganisms or organisms potentially harmful to man. The novel compounds are distinguished by excellent activity at low rates of application, by being well tolerated by plants and by being environmentally safe. They have very useful curative, preventive and systemic properties and are used for protecting numerous cultivated plants. The compounds of formula I can be used to inhibit or destroy the pests that occur on plants or parts of plants (fruit, blossoms, leaves, stems, tubers, roots) of different crops of useful plants, while at the same time protecting also those parts of the plants that grow later e.g. from phytopathogenic microorganisms.

It is also possible to use compounds of formula I as dressing agents for the treatment of plant propagation material, e.g., seed, such as fruits, tubers or grains, or plant cuttings (for example rice), for the protection against fungal infections as well as against phytopathogenic fungi occurring in the soil. The propagation material can be treated with a composition comprising a compound of formula I before planting: seed, for example, can be dressed before being sown. The active ingredients according to the invention can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

Furthermore the compounds according to present invention can be used for controlling fungi in related areas, for example in the protection of technical materials, including wood and wood related technical products, in food storage, in hygiene management.

In addition, the invention could be used to protect non-living materials from fungal attack, e.g. lumber, wall boards and paint.

The compounds of formula I are, for example, effective against the phytopathogenic fungi of the following classes: Fungi imperfecti (e.g. Alternaria spp.), Basidiomycetes (e.g. Corticium spp., Ceratobasidium spp., Waitea spp., Thanatephorus spp., Rhizoctonia spp., Hemileia spp., Puccinia spp., Phakopsora spp., Ustilago spp., Tilletia spp.), Ascomycetes (e.g. Venturia spp., Blumeria spp., Erysiphe spp., Podosphaera spp., Uncinula spp., Monilinia spp., Sclerotinia spp., Colletotrichum spp., Glomerella spp., Fusarium spp., Gibberella spp., Monographella spp., Phaeosphaeria spp., Mycosphaerella spp., Cercospora spp., Pyrenophora spp., Rhynchosporium spp., Magnaporthe spp., Gaeumannomyces spp., Oculimacula spp., Ramularia spp., Botryotinia spp.) and Oomycetes (e.g. Phytophthora spp., Pythium spp., Plasmopara spp., Peronospora spp., Pseudoperonospora spp. Bremia spp). Outstanding activity is observed against powdery mildews (e.g. Erysiphe necator) and leaf spots (e.g. Mycosphaerella spp.). Furthermore, the novel compounds of formula I are effective against phytopathogenic gram negative and gram positive bacteria (e.g. Xanthomonas spp, Pseudomonas spp, Erwinia amylovora, Ralstonia spp.) and viruses (e.g. tobacco mosaic virus).

Within the scope of present invention, target crops and/or useful plants to be protected typically comprise the following species of plants: cereal (wheat, barley, rye, oat, rice, maize, sorghum and related species); beet (sugar beet and fodder beet); pomes, drupes and soft fruit (apples, pears, plums, peaches, almonds, cherries, strawberries, raspberries and blackberries); leguminous plants (beans, lentils, peas, soybeans); oil plants (rape, mustard, poppy, olives, sunflowers, coconut, castor oil plants, cocoa beans, groundnuts); cucumber plants (pumpkins, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocado, cinnamomum, camphor) or plants such as tobacco, nuts, coffee, eggplants, sugar cane, tea, pepper, vines, hops, bananas and natural rubber plants, as well as turf and ornamentals.

The useful plants and/or target crops in accordance with the invention include conventional as well as genetically enhanced or engineered varieties such as, for example, insect resistant (e.g. Bt. and VIP varieties) as well as disease resistant, herbicide tolerant (e.g. glyphosate- and glufosinate-resistant maize varieties commercially available under the trade names RoundupReady® and LibertyLink®) and nematode tolerant varieties. By way of example, suitable genetically enhanced or engineered crop varieties include the Stoneville 5599BR cotton and Stoneville 4892BR cotton varieties.

The term “useful plants” and/or “target crops” is to be understood as including also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” and/or “target crops” is to be understood as including also useful plants which have been so transformed by the use of recombinant DNA techniques that they are capable of synthesising one or more selectively acting toxins, such as are known, for example, from toxin-producing bacteria, especially those of the genus Bacillus.

The term “useful plants” and/or “target crops” is to be understood as including also useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

The term “locus” of a useful plant as used herein is intended to embrace the place on which the useful plants are growing, where the plant propagation materials of the useful plants are sown or where the plant propagation materials of the useful plants will be placed into the soil. An example for such a locus is a field, on which crop plants are growing.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

The compounds of formula I are used in unmodified form or, preferably, together with the adjuvants conventionally employed in the art of formulation. To this end they are conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

The compounds of formula I are normally used in the form of compositions and can be applied to the crop area or plant to be treated, simultaneously or in succession with further compounds. These further compounds can be e.g. fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of formula I are normally used in the form of fungicidal compositions for controlling or protecting against phytopathogenic microorganisms, comprising as active ingredient at least one compound of formula I or of at least one preferred individual compound as above-defined, in free form or in agrochemically usable salt form, and at least one of the above-mentioned adjuvants.

Said fungicidal compositions for controlling or protecting against phytopathogenic microorganisms, comprising as active ingredient at least one compound of formula I or at least one preferred individual compound as above-defined, in free form or in agrochemically usable salt form, and at least one of the above-mentioned adjuvants can be mixed with other fungicides, resulting in some cases in unexpected synergistic activities. Mixing components which are particularly preferred are:

Azoles, such as azaconazole, BAY 14120, bitertanol, bromuconazole, cyproconazole, difenoconazole, diniconazole, epoxiconazole, fenbuconazole, fluquinconazole, flusilazole, flutriafol, hexaconazole, imazalil, imibenconazole, ipconazole, metconazole, myclobutanil, pefurazoate, penconazole, prothioconazole, pyrifenox, prochloraz, propiconazole, simeconazole, tebuconazole, tetraconazole, triadimefon, triadimenol, triflumizole, triticonazole;

Pyrimidinyl carbinoles, such as ancymidol, fenarimol, nuarimol;

2-amino-pyrimidines, such as bupirimate, dimethirimol, ethirimol;

Morpholines, such as dodemorph, fenpropidine, fenpropimorph, spiroxamine, tridemorph;

Anilinopyrimidines, such as cyprodinil, mepanipyrim, pyrimethanil;

Pyrroles, such as fenpiclonil, fludioxonil;

Phenylamides, such as benalaxyl, furalaxyl, metalaxyl, R-metalaxyl, ofurace, oxadixyl;

Benzimidazoles, such as benomyl, carbendazim, debacarb, fuberidazole, thiabendazole;

Dicarboximides, such as chlozolinate, dichlozoline, iprodione, myclozoline, procymidone, vinclozoline;

Carboxamides, such as boscalid, carboxin, fenfuram, flutolanil, mepronil, oxycarboxin, penthiopyrad, thifluzamide; guanidines, such as guazatine, dodine, iminoctadine;

Strobilurines, such as azoxystrobin, dimoxystrobin, enestroburin, fluoxastrobin, kresoxim-methyl, metominostrobin, trifloxystrobin, orysastrobin, picoxystrobin, pyraclostrobin;

Dithiocarbamates, such as ferbam, mancozeb, maneb, metiram, propineb, thiram, zineb, ziram;

N-halomethylthiotetrahydrophthalimides, such as captafol, captan, dichlofluanid, fluoromides, folpet, tolyfluanid;

Copper-compounds, such as Bordeaux mixture, copper hydroxide, copper oxychloride, copper sulfate, cuprous oxide, mancopper, oxine-copper;

Nitrophenol-derivatives, such as dinocap, nitrothal-isopropyl;

Organo-phosphorus-derivatives, such as edifenphos, iprobenphos, isoprothiolane, phosdiphen, pyrazophos, tolclofos-methyl;

Triazolopyrimidine derivatives which are known and may be prepared by methods as described in WO98/46607, such as 5-chloro-7-(4-methyl-piperidin-1-yl)-6-(2,4,6-trifluoro-phenyl)-[1,2,4]triazolo[1,5-a]pyrimidine (formula T.1);

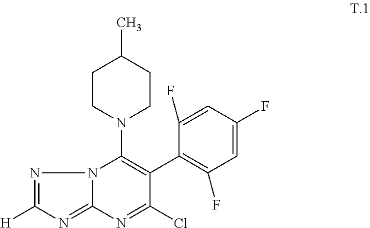

Carboxamide derivatives which are known and may be prepared by methods as described in WO04/035589, WO06/37632, WO03/074491 or WO03070705, such as 3-difluoromethyl-1-methyl-1H-pyrazole-4-carboxylic acid (9-isopropyp-1,2,3,4-tetrahaydro-1,4-methano-naphthalen-5-yl)-amide (formula U.1), 3-difluoromethyl-1-methyl-1H-pyrazole-4-carboxylic acid (2-bicyclopropyl-2-yl-phenyl)-amide (formula U.2) or N-(3′,4′-dichloro-5-fluoro-1,1′-biphenyl-2-yl)-3-(difluoromethyl)-1-methyl-1H-pyrazole-4-carboxamide;

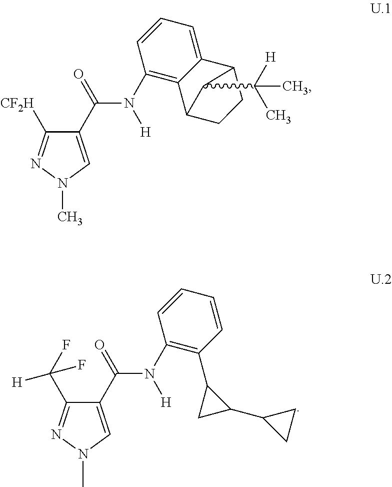

Benzamide derivatives which are known and may be prepared by methods as described in WO 2004/016088, such as N-{-2-[3-chloro-5-(trifluoromethyl)-2-pyridinyl]ethyl}-2-trifluoromethylbenzamide, which is also known under the name fluopyram (formula V.1);

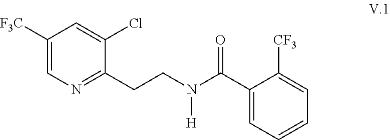

and

Various others, such as acibenzolar-S-methyl, anilazine, benthiavalicarb, blasticidin-S, chinomethionate, chloroneb, chlorothalonil, cyflufenamid, cymoxanil, dichlone, diclocymet, diclomezine, dicloran, diethofencarb, dimethomorph, flumorph, dithianon, ethaboxam, etridiazole, famoxadone, fenamidone, fenoxanil, fentin, ferimzone, fluazinam, fluopicolide, flusulfamide, fenhexamid, fosetyl-aluminium, hymexazol, iprovalicarb, cyazofamid, kasugamycin, mandipropamid, methasulfocarb, metrafenone, nicobifen, pencycuron, phthalide, polyoxins, probenazole, propamocarb, proquinazid, pyroquilon, quinoxyfen, quintozene, sulfur, tiadinil, triazoxide, tricyclazole, triforine, validamycin, zoxamide and glyphosate.

Another aspect of invention is related to the use of a compound of formula I or of a preferred individual compound as above-defined, of a composition comprising at least one compound of formula I or at least one preferred individual compound as above-defined, or of a fungicidal mixture comprising at least one compound of formula I or at least one preferred individual compound as above-defined, in admixture with other fungicides, as described above, for controlling or preventing infestation of plants, harvested food crops, seeds or non-living materials by phytopathogenic microorganisms, preferably fungal organisms.

A further aspect of invention is related to a method of controlling or preventing an infestation of crop plants, harvested food crops or of non-living materials by phytopathogenic or spoilage microorganisms or organisms potentially harmful to man, especially fungal organisms, which comprises the application of a compound of formula I or of a preferred individual compound as above-defined as active ingredient to the plants, to parts of the plants or to the locus thereof, to seeds or to any part of the non-living materials.

Controlling or preventing means reducing the infestation of crop plants or of non-living materials by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, which comprises the application of a compound of formula I, or an agrochemical composition which contains at least one of said compounds, is foliar application. The frequency of application and the rate of application will depend on the risk of infestation by the corresponding pathogen. However, the compounds of formula I can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula I may also be applied to seeds (coating) by impregnating the seeds or tubers either with a liquid formulation of the fungicide or coating them with a solid formulation.

A formulation [that is, a composition containing the compound of formula I] and, if desired, a solid or liquid adjuvant or monomers for encapsulating the compound of formula I, is prepared in a known manner, typically by intimately mixing and/or grinding the compound with extenders, for example solvents, solid carriers and, optionally, surface active compounds (surfactants).

The agrochemical formulations will usually contain from 0.1 to 99% by weight, preferably from 0.1 to 95% by weight, of the compound of formula I, 99.9 to 1% by weight, preferably 99.8 to 5% by weight, of a solid or liquid adjuvant, and from 0 to 25% by weight, preferably from 0.1 to 25% by weight, of a surfactant.

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

Whereas it is preferred to formulate commercial products as concentrates, the end user will normally use dilute formulations.

Plant growth regulators (PGRs) are generally any substances or mixtures of substances intended to accelerate or retard the rate of growth or maturation, or otherwise alter the development of plants or their produce.

Plant growth regulators (PGRs) affect growth and differentiation of plants.

More specifically, various plant growth regulators (PGRs) can, for example, reduce plant height, stimulate seed germination, induce flowering, darken leaf coloring, change the rate of plant growth and modify the timing and efficiency of fruiting.

Furthermore, the present invention also relates to compositions comprising the novel imidazole derivatives of the present invention that improve plants, a process which is commonly and hereinafter referred to as “plant health”.

For example, advantageous properties that may be mentioned are improved crop characteristics including: emergence, crop yields, protein content, increased vigour, faster maturation, increased speed of seed emergence, improved nitrogen utilization efficiency, improved water use efficiency, improved oil content and for quality, improved digestibility, faster ripening, improved flavor, improved starch content, more developed root system (improved root growth), improved stress tolerance (e.g. against drought, heat, salt, light, UV, water, cold), reduced ethylene (reduced production and/or inhibition of reception), tillering increase, increase in plant height, bigger leaf blade, less dead basal leaves, stronger tillers, greener leaf color, pigment content, photosynthetic activity, less input needed (such as fertilizers or water), less seeds needed, more productive tillers, earlier flowering, early grain maturity, less plant verse (lodging), increased shoot growth, enhanced plant vigor, increased plant stand and early and better germination.

Advantageous properties, obtained especially from treaded seeds, are e.g. improved germination and field establishment, better vigor, more homogeneous field establishment.

Advantageous properties, obtained especially from foliar and/or in-furrow application are e.g. improved plant growth and plant development, better growth, more tillers, greener leaves, larger leaves, more biomass, better roots, improved stress tolerance of the plants, more grain yield, more biomass harvested, improved quality of the harvest (content of fatty acids, metabolites, oil etc), more marketable products (e.g. improved size), improved process (e.g. longer shelf-life, better extraction of compounds), improved quality of seeds (for being seeded in the following seasons for seed production); or any other advantages familiar to a person skilled in the art.

It is therefore an object of the present invention to provide a method which solves the problems outlined above.

The present invention relates to plant-protecting active ingredients that are imidazole compounds of formula I according to the invention, in particular the individual imidazole compounds described in the above description as being preferred, and mixtures with increased efficacy and to a method of improving the health of plants by applying said compounds and mixtures to the plants or the locus thereof.

The action of the compounds of formula I goes beyond the known fungicidal action. The imidazole compounds of formula I according to the invention, in particular the individual imidazole compounds described in the above description as being preferred compounds exhibit plant health

The term plant health comprises various sorts of improvements of plants that are not connected to the control of harmful fungi.

In another aspect, the present invention relates to a composition comprising at least one compound a compound of formula I or of a preferred individual compound as above-defined and/or at least one pharmaceutically acceptable salt thereof, at least one pharmaceutically acceptable carrier and/or at least one pharmaceutically acceptable diluent.

In a further aspect, the present invention also relates to a compound of formula I or of a preferred individual compound as above-defined, or a pharmaceutically acceptable salt thereof for use as a medicament.

In a preferred aspect, the present invention also relates to a compound of formula I or of a preferred individual compound as above-defined, or a pharmaceutically acceptable salt thereof for the treatment of cancer.

In an additional aspect, the present invention also relates to the use of a compound formula I or of a preferred individual compound as above-defined, or a pharmaceutically acceptable salt thereof in the manufacture of a medicament for the treatment of cancer.

In a particular aspect, the present invention also relates to a method of treating cancer in a subject in need thereof, comprising administering a compound formula I or of a preferred individual compound as above-defined to said subject in an amount effective to treat said cancer.

The invention further provides fungicidal or pharmaceutical compositions comprising these compounds I and/or their agriculturally or pharmaceutically acceptable salts and suitable carriers.

Suitable pharmaceutically acceptable carriers are described below.

The imidazole compounds of formula I according to the invention, in particular the imidazoles of formula I according to the invention described in the above description as being preferred, and/or their pharmaceutically acceptable salts are suitable for the treatment, inhibition or control of growth and/or propagation of tumor cells and the disorders associated therewith.

Accordingly, they are suitable for cancer therapy in warmblooded vertebrates, for example mammals and birds, in particular man, but also other mammals, in particular useful and domestic animals, such as dogs, cats, pigs, ruminants (cattle, sheep, goats, bison, etc.), horses and birds, such as chicken, turkey, ducks, geese, guineafowl and the like.

The imidazoles of formula I according to the invention, in particular the imidazoles of formula I according to the invention described in the above description as being preferred, and/or their pharmaceutically acceptable salts are suitable for the therapy of cancer or cancerous disorders of the following organs: breast, lung, intestine, prostate, skin (melanoma), kidney, bladder, mouth, larynx, esophagus, stomach, ovaries, pancreas, liver and brain.

In addition to the imidazole compounds of formula I according to the invention and/or its pharmaceutically acceptable salt, the pharmaceutical compositions according to the invention comprise at least optionally a suitable carrier.

“Pharmaceutically acceptable” means compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Suitable carriers are, for example, solvents, carriers, excipients, binders and the like customarily used for pharmaceutical formulations, which are described below in an exemplary manner for individual types of administration.

“Pharmaceutically acceptable carrier” as used herein means a pharmaceutically-acceptable material, composition or vehicle, such as a liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include:
sugars, such as lactose, glucose and sucrose;
starches, such as corn starch and potato starch;
cellulose, and its derivatives, such as sodium carboxymethyl cellulose, ethyl cellulose and cellulose acetate;
powdered tragacanth;
malt;
gelatin;
talc;
excipients, such as cocoa butter and suppository waxes;
oils, such as peanut oil, cottonseed oil, safflower oil, sesame oil, olive oil, corn oil and soybean oil;
glycols, such as propylene glycol;
polyols, such as glycerin, sorbitol, mannitol and polyethylene glycol;
esters, such as ethyl oleate and ethyl laurate;
agar; buffering agents, such as magnesium hydroxide and aluminum hydroxide;
alginic acid;
pyrogen-free water;
isotonic saline;
Ringer's solution;
ethyl alcohol;
phosphate buffer solutions; and
other non-toxic compatible substances employed in pharmaceutical formulations.

The imidazole compounds of formula I according to the invention, in particular the individual imidazole compounds described in the above description as being preferred (the active compound), can be administered in a customary manner, for example orally, intravenously, intramuscularly or subcutaneously.

For oral administration, the active compound can be mixed, for example, with an inert diluent or with an edible carrier; it can be embedded into a hard or soft gelatin capsule, it can be compressed to tablets or it can be mixed directly with the food/feed.

The active compound can be mixed with excipients and administered in the form of indigestible tablets, buccal tablets, pastilles, pills, capsules, suspensions, potions, syrups and the like.

Such preparations should contain at least 0.1% of active compound.

The composition of the preparation may, of course, vary.

It usually comprises from 2 to 60% by weight of active compound, based on the total weight of the preparation in question (dosage unit).

Preferred preparations of the imidazole compounds of formula I according to the invention, in particular the individual imidazole compounds described in the above description as being preferred, comprise from 10 to 1000 mg of active compound per oral dosage unit.

The tablets, pastilles, pills, capsules and the like may furthermore comprise the following components: binders, such as traganth, gum arabic, corn starch or gelatin, excipients, such as dicalcium phosphate, disintegrants, such as corn starch, potato starch, alginic acid and the like, glidants, such as magnesium stearate, sweeteners, such as sucrose, lactose or saccharin, and/or flavors, such as peppermint, vanilla and the like.

Capsules may furthermore comprise a liquid carrier.

Other substances which modify the properties of the dosage unit may also be used.

For example, tablets, pills and capsules may be coated with schellack, sugar or mixtures thereof.

In addition to the active compound, syrups or potions may also comprise sugar (or other sweeteners), methyl- or propylparaben as preservative, a colorant and/or a flavor.

The components of the active compound preparations must, of course, be pharmaceutically pure and nontoxic at the quantities employed.

Furthermore, the active compounds can be formulated as preparations with a controlled release of active compound, for example as delayed-release preparations.

The active compounds can also be administered parenterally or intraperitoneally.

Solutions or suspensions of the active compounds or their salts can be prepared with water using suitable wetting agents, such as hydroxypropylcellulose.

Dispersions can also be prepared using glycerol, liquid polyethylene glycols and mixtures thereof in oils.

Frequently, these preparations furthermore comprise a preservative to prevent the growth of microorganisms.

Preparations intended for injections comprise sterile aqueous solutions and dispersions and also sterile powders for preparing sterile solutions and dispersions.

The preparation has to be sufficiently liquid for injection.

It has to be stable under the preparation and storage conditions and it has to be protected against contamination by microorganisms.

The carrier may be a solvent or a dispersion medium, for example, water, ethanol, a polyol (for example glycerol, propylene glycol or liquid polyethylene glycol), a mixture thereof and/or a vegetable oil.

Pharmaceutical compositions of this invention suitable for parenteral administration comprise a compound of formula I in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. These compositions may also contain adjuvants such as preservatives, wetting agents, emulsifying agents and dispersing agents. Prevention of the action of microorganisms may be ensured by the inclusion of various antibacterial and other antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The pharmaceutical compositions of the present invention may be given by any suitable means of administration including orally, parenterally, topically, transdermally, rectally, etc. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Topical or parenteral administration is preferred.

The following non-limiting examples illustrate the above-described invention in more detail.

### EXAMPLE 1

### This Example Illustrates the Preparation of 1-[2,5-dichloro-3-(2,4,6-trifluoro-phenyl)-3H-imidazol-4-yl]-4-methyl-piperidine (Compound No I.a.003.)

### a) Preparation of 1-[2-(4-methyl-piperidin-1-yl)-2-oxo-ethyl]-3-(2,4,6-trifluoro-phenyl)-thiourea

To a solution of 2-amino-1-(4-methyl-piperidin-1-yl)-ethanone hydrochloride salt (11 g) in dichloromethane (385 ml) are added successively N,N-diisopropylethylamine (19.5 ml) and 2,4,6-trifluorophenyl isothiocyanate (11.13 g). After 1 hour stirring at room temperature, the solvent is evaporated and the residue is purified by chromatography column on silica gel, using a mixture of heptane/ethyl acetate 1:1 as eluent to obtain 13.6 g of 1-[2-(4-methyl-piperidin-1-yl)-2-oxo-ethyl]-3-(2,4,6-trifluoro-phenyl)-thiourea. ¹H NMR (300 Mhz, CDCl₃) 8.1 ppm, 1H, bs; 7.8 ppm, 1H, bs; 6.78 ppm, 2H, t, J=7.8 Hz; 4.4 ppm, 2H, bs; 4.3 ppm, 1H, bd, J=13.2 Hz; 3.75 ppm, 1H, bd, J=13. and 6 Hz; 3 ppm, dt, 1H, J=2.5, 13 Hz; 2.6 ppm, 1H, dt, J=2.5, 13 Hz; 1.74 ppm, 1H, bd, J=13.1 Hz; 1.68-1.52 ppm, 2H, m; 1.14-0.95 ppm, 2H, m; 0.94 ppm, 3H, d, J=6.4 Hz.

### b) Preparation of 2-methyl-1-[2-(4-methyl-piperidin-1-yl)-2-oxo-ethyl]-3-(2,4,6-trifluoro-phenyl)-isothiourea

To a solution of 1-[2-(4-methyl-piperidin-1-yl)-2-oxo-ethyl]-3-(2,4,6-trifluoro-phenyl)-thiourea (13.6 g) in acetonitrile (136 ml) is added successively anhydrous potassium carbonate (8.2 g) iodomethane (4.9 ml). After one hour stirring at room temperature, the solvent is evaporated; the residue is diluted in ethyl acetate and washed with water. The aqueous phase is extracted twice with ethyl acetate, the combined organic phases are washed once with water and once with brine, before being dried over sodium sulphate, filtered and the solvent evaporated under reduced pressure. The residue is purified by chromatography column on silica gel, using a mixture of heptane/ethyl acetate 1:1 as eluent to obtain 13.6 g of 2-methyl-1-[2-(4-methyl-piperidin-1-yl)-2-oxo-ethyl]-3-(2,4,6-trifluoro-phenyl)-isothiourea. ¹H NMR (300 Mhz, CDCl₃) 6.66 ppm, 2H, t, J=8.2 Hz; 6.28 ppm, 1H, bs; 4.55 ppm, 1H, bd, J=13 Hz; 4.22 ppm, 2H, bs; 3.72 ppm, 1H, bd, J=13.6 Hz; 3 ppm, dt, 1H, J=2.5, 13 Hz; 2.67 ppm, 1H, dt, J=2.5, 13 Hz; 2.44 ppm, 3H, s; 1.72 ppm, 1H, bd, J=13.4 Hz; 1.71-1.54 ppm, 1H, m; 1.11 ppm, 2H, dq, J=2.5, 13.2 Hz; 0.97 ppm, 3H, d, J=6.4 Hz.

### c) Preparation of 4-methyl-1-[2-methylsulfanyl-3-(2,4,6-trifluoro-phenyl)-3H-imidazol-4-yl]-piperidine

To a green solution of 2-methyl-1-[2-(4-methyl-piperidin-1-yl)-2-oxo-ethyl]-3-(2,4,6-trifluoro-phenyl)-isothiourea (13.6 g) in dimethoxyethane (136 ml) is added 2,4-bis(4-methoxyphenyl)-1,3,2,4-dithiadiphosphetane 2,4-disulfide (8.6 g). The reaction turns red and is stirred overnight at room temperature. The solvent is then evaporated, and the residual purple oil (19.93 g) is diluted with tert-butyl methyl ether, the precipitate is filtered, the filtrate is washed with 4N NaOH. The color changes from purple to blue, while the phases are stirred for about 30 minutes. The aqueous phase is extracted twice with ethyl acetate, the combined organic phases are washed with brine, dried over sodium sulphate, filtered and the solvent are evaporated under reduced pressure. The residue is purified by chromatography column on silica gel (520 g), using a mixture of heptane/ethyl acetate 7:3 as eluent to obtain 4.54 g of 4-methyl-1-[2-methylsulfanyl-3-(2,4,6-trifluoro-phenyl)-3H-imidazol-4-yl]-piperidine. ¹H NMR (300 Mhz, CDCl₃) 6.85 ppm, 2H, t, J=7.88 Hz; 6.72 ppm, 1H, s; 2.91 ppm, 2H, bd, J=12 Hz; 2.59 ppm, 2H, dt, J=2.5, 11.9 Hz; 2.48 ppm, 3H, s; 1.52 ppm, 2H, bd, J=12.8 Hz; 1.45-1.3 ppm, 1H, m; 0.99 ppm, 2H, dq, J=4, 12 Hz; 0.86 ppm, 3H, d, J=6.5 Hz.

### d) Preparation of 1-[5-chloro-2-methylsulfanyl-3-(2,4,6-trifluoro-phenyl)-3H-imidazol-4-yl]-4-methyl-piperidine

To a solution of 4-methyl-1-[2-methylsulfanyl-3-(2,4,6-trifluoro-phenyl)-3H-imidazol-4-yl]-piperidine (1 g) in chloroform (12 ml) is added N-chlorosuccinimide (0.468 g). The reaction is stirred 1 hour at room temperature, before being diluted with dichloromethane and cold 1N NaOH. The aqueous phase is extracted three times with dichloromethane, the combined organic extracts are washed with brine, dried over sodium sulphate, filtered and the solvents are evaporated. The residual 1.1 g of brown oil is purified by chromatography column on silica gel (50 g), using a mixture of heptane/ethyl acetate 9:1 as eluent to obtain 0.722 g of 1-[5-chloro-2-methylsulfanyl-3-(2,4,6-trifluoro-phenyl)-3H-imidazol-4-yl]-4-methyl-piperidine. ¹H NMR (300 Mhz, CDCl₃) 6.85 ppm, 2H, t, J=7.88 Hz; 3 ppm, 2H, dt, J=2.4, 11.7 Hz; 2.86 ppm, 2H, bd, J=11.5 Hz; 2.52 ppm, 3H, s; 1.51 ppm, 2H, bd, J=12.3 Hz; 1.45-1.3 ppm, 1H, m; 0.86-0.72 ppm, 2H, m; 0.85 ppm, 3H, d, J=6.5 Hz.

### e) Preparation of 1-[5-chloro-2-methanesulfonyl-3-(2,4,6-trifluoro-phenyl)-3H-imidazol-yl]-4-methyl-piperidine

To a cooled (zero degree) solution of 1-[5-chloro-2-methylsulfanyl-3-(2,4,6-trifluoro-phenyl)-3H-imidazol-4-yl]-4-methyl-piperidine (0.722 g) in dichloromethane (14.44 ml) is added meta-chloroperbenzoic acid (1.113 g). The reaction mixture is allowed to warm up to room temperature after 10 minutes, and is stirred overnight. As the reaction is not complete after 16 hours, 113 mg of meta-chloroperbenzoic acid are added and, after 4 hours, ice and a 10% thiosulfate solution are added and the reaction stirred vigorously. The organic phase is washed twice with a 10% thiosulfate solution, twice with a saturated bicarbonate solution and twice with brine. The organic phase is then dried over sodium sulphate, filtered and the solvents are evaporated. 0.735 g of 1-[5-chloro-2-methanesulfonyl-3-(2,4,6-trifluoro-phenyl)-3H-imidazol-yl]-4-methyl-piperidine are then obtained. ¹H NMR (300 Mhz, CDCl₃) 6.85 ppm, 2H, t, J=7.88 Hz; 3 ppm, 2H, dt, J=2.4, 11.7 Hz; 2.86 ppm, 2H, bd, J=11.5 Hz; 2.52 ppm, 3H, s; 1.51 ppm, 2H, bd, J=12.3 Hz; 1.45-1.3 ppm, 1H, m; 0.86-0.72 ppm, 2H, m; 0.85 ppm, 3H, d, J=6.5 Hz.

### f) Preparation of 1-[5-chloro-3-(2,4,6-trifluoro-phenyl)-3H-imidazol-4-yl]-4-methyl-piperidine

To a solution of 1-[5-chloro-2-methanesulfonyl-3-(2,4,6-trifluoro-phenyl)-3H-imidazol-yl]-4-methyl-piperidine (350 mg) in dry THF (17.5 ml) is added dry methanol (17.5 ml). After cooling to −35° C., Na₂HPO₄ (0.305 g) and Na(Hg) 5% (1.535 g) are successively added. The reaction mixture is stirred at this temperature for 5 hours before being allowed to warm up at room temperature. The reaction mixture is then filtered onto a pad of Celite, the filtrate is acidified with 2 ml of 1N HCl and then evaporated. The residue is taken up in water and ethyl acetate, the water phase is extracted twice with ethyl acetate. The combined organic extracts are washed once with water and once with brine, then dried over sodium sulphate, filtered and the solvents are evaporated. The residual 0.296 g of yellow oil is purified by chromatography column on silica gel, using a mixture of heptane/ethyl acetate 4:1 as eluent to obtain 0.207 g of 1-[5-chloro-3-(2,4,6-trifluoro-phenyl)-3H-imidazol-4-yl]-4-methyl-piperidine. ¹H NMR (300 Mhz, CDCl₃) 7.22 ppm, 1H, s; 6.85 ppm, 2H, t, J=9.5 Hz; 3.07 ppm, 2H, dt, J=2.3, 11.7 Hz; 2.86 ppm, 2H, bd, J=11.6 Hz; 1.54 ppm, 2H, bd, J=12.6 Hz; 1.45-1.25 ppm, 1H, m; 0.90 ppm, 2H, dq, J=4, 12 Hz; 0.86 ppm, 3H, d, J=6.5 Hz.

### g) Preparation of 1-[2,5-dichloro-3-(2,4,6-trifluoro-phenyl)-3H-imidazol-4-yl]-4-methyl-piperidine (Compound No I.a.003.)

To a solution of 1-[5-chloro-3-(2,4,6-trifluoro-phenyl)-3H-imidazol-4-yl]-4-methyl-piperidine (0.2 g) in chloroform (3 ml) is added N-chlorosuccinimide (0.130 g). The reaction mixture is stirred at 60° C. for 1 hour, before being allowed to cool down to room temperature. The reaction mixture is diluted with dichloromethane and 1N sodium Hydroxide. The aqueous phase is extracted three times with dichloromethane, the combined organic extracts is washed with 1N NaOH and brine, dried over sodium sulphate, filtered and the solvents are evaporated. The residual solid is purified by chromatography column on silica gel, using a mixture of heptane/ethyl acetate 9:1 as eluent to obtain 0.064 g of 1-[2,5-dichloro-3-(2,4,6-trifluoro-phenyl)-3H-imidazol-4-yl]-4-methyl-piperidine (Compound No I.a.003.) as a white solid. ¹H NMR (300 Mhz, CDCl₃) 6.87 ppm, 2H, t, J=7.7 Hz; 3.03 ppm, 2H, dt, J=1.9, 11.7 Hz; 2.86 ppm, 2H, bd, J=11.5 Hz; 1.53 ppm, 2H, bd, J=12.4 Hz; 1.48-1.32 ppm, 1H, m; 0.92-0.82 ppm, 2H, m; 0.86 ppm, 3H, d, J=6.5 Hz.

### EXAMPLE 2

### This Example Illustrates the Preparation of 1-[2-chloro-3-(2,6-difluoro-4-methoxy-phenyl)-5-methyl-3H-imidazol-4-yl]-4-methyl-piperidine (Compound No I.b.004.)

a) Preparation of 1-[5-bromo-2-methylsulfanyl-3-(2,4,6-trifluoro-phenyl)-3H-imidazol-4-yl]-4-methyl-piperidine

To a solution of 4-methyl-1-[2-methylsulfanyl-3-(2,4,6-trifluoro-phenyl)-3H-imidazol-4-yl]-piperidine (2.07 g) in chloroform (25 ml) is added N-bromosuccinimide (1.4 g). The reaction mixture is stirred at room temperature for 10 minutes. The reaction mixture is diluted with dichloromethane and 1N sodium hydroxide. The aqueous phase is extracted three times with dichloromethane, the combined organic extracts is washed with 1N NaOH and brine, dried over sodium sulphate, filtered and the solvents are evaporated. The residual solid is purified by chromatography column on silica gel, using a mixture of heptane/ethyl acetate 9:1 as eluent to obtain 1.53 g of 1-[5-bromo-2-methylsulfanyl-3-(2,4,6-trifluoro-phenyl)-3H-imidazol-4-yl]-4-methyl-piperidine. ¹H NMR (300 Mhz, CDCl₃) 6.85 ppm, 2H, t, J=7.8 Hz; 3.06 ppm, 2H, dt, J=2.2, 11.7 Hz; 2.84 ppm, 2H, bd, J=11.6 Hz; 2.53 ppm, 3H, s; 1.52 ppm, 2H, bd, J=11.8 Hz; 1.35 ppm, 1H, m; 0.89-0.79 ppm, 2H, m; 0.85 ppm, 3H, d, J=6.41 Hz.

### b) Preparation of 4-methyl-1-[5-methyl-2-methylsulfanyl-3-(2,4,6-trifluoro-phenyl)-3H-imidazol-4-yl]-piperidine

To a solution of 1-[5-bromo-2-methylsulfanyl-3-(2,4,6-trifluoro-phenyl)-3H-imidazol-4-yl]-4-methyl-piperidine (1.53 g) in dioxane (31 ml) is added cesium carbonate (4.74 g), trimethylboroxine (0.66 g) and PdCl₂(dppf)₂.CH₂Cl₂ (0.89 g). The mixture is then heated at 90° C. overnight. After being cooled down to room temperature, the mixture is filtered and the filtrate is washed with water. The aqueous phase is extracted twice with ethyl acetate, the combined organic extracts are washed with brine, dried over sodium sulphate, filtered and the solvents are evaporated. The residual mixture is purified by chromatography column on silica gel, using a mixture of heptane/ethyl acetate 4:1 as eluent to obtain 0.552 g of 4-methyl-1-[5-methyl-2-methylsulfanyl-3-(2,4,6-trifluoro-phenyl)-3H-imidazol-4-yl]-piperidine. ¹H NMR (300 Mhz, CDCl₃) 6.83 ppm, 2H, t, J=7.85 Hz; 2.84 ppm, 4H, m; 2.47 ppm, 3H, s; 2.26 ppm, 3H, s; 1.49 ppm, 2H, bd, J=12 Hz; 1.36-1.24 ppm, 1H, m; 0.91-0.81 ppm, 2H, m; 0.85 ppm, 3H, d, J=6.6 Hz.

### c) Preparation of 1-[3-(2,6-Difluoro-4-methoxy-phenyl)-5-methyl-2-methylsulfanyl-3H-imidazol-4-yl]-4-methyl-piperidine

To a solution of 4-methyl-1-[5-methyl-2-methylsulfanyl-3-(2,4,6-trifluoro-phenyl)-3H-imidazol-4-yl]-piperidine (0.625 g) in methanol (6.2 ml) is added three equivalents of sodium methanolate. The reaction mixture is then heated to reflux for 30 minutes, then cooled down to room temperature. It is poured onto cold 1N HCl, and the aqueous phase is extracted twice with ethyl acetate, the combined organic extracts are washed with brine, dried over sodium sulphate, filtered and the solvents are evaporated. The residual mixture is purified by chromatography column on silica gel, using a mixture of heptane/ethyl acetate 3:2 as eluent to obtain 0.263 g of 1-[3-(2,6-difluoro-4-methoxy-phenyl)-5-methyl-2-methylsulfanyl-3H-imidazol-4-yl]-4-methyl-piperidine. ¹H NMR (300 Mhz, CDCl₃) 6.57 ppm, 2H, d, J=8.9 Hz; 3.86 ppm, 3H, s; 2.91-2.79 ppm, 4H, m; 2.47 ppm, 3H, s; 2.25 ppm, 3H, s: 1.49 ppm, 2H, bd, J=12.1 Hz; 1.34-1.25 ppm, 1H, m; 0.94-0.87 ppm, 2H, m; 0.84 ppm, 3H, d, J=6.6 Hz.

### d) Preparation of 1-[3-(2,6-difluoro-4-methoxy-phenyl)-5-methyl-3H-imidazol-4-yl]-4-methyl-piperidine

To a solution of 1-[3-(2,6-difluoro-4-methoxy-phenyl)-5-methyl-2-methylsulfanyl-3H-imidazol-4-yl]-4-methyl-piperidine (0.226 g) in ethanol (4.5 ml) is added 22.64 ml of an aqueous Raney-Nickel suspension (100 g/l). The black suspension is heated to reflux for 90 minutes, before being cooled down to room temperature. Celite is added to the mixture and the suspension is filtered over a celite pad. The celite is rinsed with ethanol and water, taking care that the pad always stays “wet”. The filtrate is then concentrated under reduced pressure, diluted with ethyl acetate and washed with water. The aqueous phase is extracted twice with ethyl acetate, the combined organic extracts are washed with brine, dried over sodium sulphate, filtered and the solvents are evaporated. The residual mixture is purified by chromatography column on silica gel, using a mixture of heptane/ethyl acetate 1:4 as eluent to obtain 0.067 g of 1-[3-(2,6-difluoro-4-methoxy-phenyl)-5-methyl-3H-imidazol-4-yl]-4-methyl-piperidine. ¹H NMR (300 Mhz, CDCl₃) 7.22 ppm, 1H, s; 6.56 ppm, 2H, d, J=8.9 Hz; 3.85 ppm, 3H, s; 2.91-2.83 ppm, 4H, m; 2.26 ppm, 3H, s: 1.49 ppm, 2H, bd, J=12.1 Hz; 1.37-1.30 ppm, 1H, m; 0.98-0.88 ppm, 2H, m; 0.86 ppm, 3H, d, J=6.5 Hz.

### e) Preparation of 1-[2-chloro-3-(2,6-difluoro-4-methoxy-phenyl)-5-methyl-3H-imidazol-4-yl]-4-methyl-piperidine (Compound No I.b.004.)

To a solution of 1-[3-(2,6-difluoro-4-methoxy-phenyl)-5-methyl-3H-imidazol-4-yl]-4-methyl-piperidine (0.067 g) in chloroform (1.3 ml) is added N-chlorosuccinimide (0.035 g). The reaction mixture is stirred at 0° C. for 90 minutes. The reaction mixture is concentrated under reduced pressure, then diluted with methanol. Isolute is added, and the methanol removed under reduced pressure. The residual solid is purified by chromatography column on silica gel, using a mixture of heptane/ethyl acetate 4:1 as eluent to obtain 0.014 g of 1-[2-chloro-3-(2,6-difluoro-4-methoxy-phenyl)-5-methyl-3H-imidazol-4-yl]-4-methyl-piperidine (Compound No I.b.004.). ¹H NMR (300 Mhz, CDCl₃) 6.59 ppm, 2H, d, J=8.9 Hz; 3.87 ppm, 3H, s; 2.90 ppm, 2H, bd, J=11.2 Hz; 2.82 ppm, 2H, dt, J=2.3, 11.5 Hz; 2.21 ppm, 3H, s: 1.49 ppm, 2H, bd, J=12.2 Hz; 1.35-1.25 ppm, 1H, m; 0.95-0.88 ppm, 2H, m; 0.85 ppm, 3H, d, J=6.6 Hz

Table 1 below illustrates examples of individual compounds of formula I according to the invention.

TABLE 1
individual compounds of formula I according to the invention

| Compound | | | | |
|---|---|---|---|---|
| No. | R¹ | R² | R³ | R⁵ |
| 001 | Cl | —(CH₂)₂—CH(CH₃)—(CH₂)₂— | | F |
| 002 | CH₃ | —(CH₂)₂—CH(CH₃)—(CH₂)₂— | | F |
| 003 | Cl | —(CH₂)₂—CH(CH₃)—(CH₂)₂— | | Cl |
| 004 | CH₃ | —(CH₂)₂—CH(CH₃)—(CH₂)₂— | | Cl |
| 005 | Cl | —CH(CH₃)—(CH₂)₄— | | F |
| 006 | CH₃ | —CH(CH₃)—(CH₂)₄— | | F |
| 007 | Cl | —CH(CH₃)—(CH₂)₄— | | Cl |
| 008 | CH₃ | —CH(CH₃)—(CH₂)₄— | | Cl |
| 009 | Cl | —CH═CH—(CH₂)₃— | | F |
| 010 | CH₃ | —CH═CH—(CH₂)₃— | | F |
| 011 | Cl | —CH═CH—(CH₂)₃— | | Cl |
| 012 | CH₃ | —CH═CH—(CH₂)₃— | | Cl |
| 013 | Cl | —CH₂—CH═CH—(CH₂)₂— | | F |
| 014 | CH₃ | —CH₂—CH═CH—(CH₂)₂— | | F |
| 015 | Cl | —CH₂—CH═CH—(CH₂)₂— | | Cl |
| 016 | CH₃ | —CH₂—CH═CH—(CH₂)₂— | | Cl |
| 017 | Cl | —CH₂—CH(OH)—(CH₂)₃— | | F |
| 018 | CH₃ | —CH₂—CH(OH)—(CH₂)₃— | | F |
| 019 | Cl | —CH₂—CH(OH)—(CH₂)₃— | | Cl |
| 020 | CH₃ | —CH₂—CH(OH)—(CH₂)₃— | | Cl |
| 021 | Cl | —(CH₂)₄— | | F |
| 022 | CH₃ | —(CH₂)₄— | | F |
| 023 | Cl | —(CH₂)₄— | | Cl |
| 024 | CH₃ | —(CH₂)₄— | | Cl |
| 025 | Cl | —(CH₂)₅— | | F |
| 026 | CH₃ | —(CH₂)₅— | | F |
| 027 | Cl | —(CH₂)₅— | | Cl |
| 028 | CH₃ | —(CH₂)₅— | | Cl |
| 029 | Cl | —(CH₂)₆— | | F |
| 030 | CH₃ | —(CH₂)₆— | | F |
| 031 | Cl | —(CH₂)₆— | | Cl |
| 032 | CH₃ | —(CH₂)₆— | | Cl |
| 033 | Cl | —(CH₂)₂—O—(CH₂)₂— | | F |
| 034 | CH₃ | —(CH₂)₂—O—(CH₂)₂— | | F |
| 035 | Cl | —(CH₂)₂—O—(CH₂)₂— | | Cl |
| 036 | CH₃ | —(CH₂)₂—O—(CH₂)₂— | | Cl |
| 037 | Cl | —(CH₂)₂—S—(CH₂)₂— | | F |
| 038 | CH₃ | —(CH₂)₂—S—(CH₂)₂— | | F |
| 039 | Cl | —(CH₂)₂—S—(CH₂)₂— | | Cl |
| 040 | CH₃ | —(CH₂)₂—S—(CH₂)₂— | | Cl |
| 049 | Cl | ethyl | ethyl | F |
| 050 | CH₃ | ethyl | ethyl | F |
| 051 | Cl | ethyl | ethyl | Cl |
| 052 | CH₃ | ethyl | ethyl | Cl |
| 053 | Cl | 2,2,2-trifluoroethyl | ethyl | F |
| 054 | CH₃ | 2,2,2-trifluoroethyl | ethyl | F |
| 055 | Cl | 2,2,2-trifluoroethyl | ethyl | Cl |
| 056 | CH₃ | 2,2,2-trifluoroethyl | ethyl | Cl |
| 057 | Cl | ethyl | H | F |
| 058 | CH₃ | ethyl | H | F |
| 059 | Cl | ethyl | H | Cl |
| 060 | CH₃ | ethyl | H | Cl |
| 061 | Cl | 2,2,2-trifluoroethyl | H | F |
| 062 | CH₃ | 2,2,2-trifluoroethyl | H | F |
| 063 | Cl | 2,2,2-trifluoroethyl | H | Cl |
| 064 | CH₃ | 2,2,2-trifluoroethyl | H | Cl |
| 065 | Cl | 2-(1,1,1-trifluoropropyl) | H | F |
| 066 | CH₃ | 2-(1,1,1-trifluoropropyl) | H | F |
| 067 | Cl | 2-(1,1,1-trifluoropropyl) | H | Cl |
| 068 | CH₃ | 2-(1,1,1-trifluoropropyl) | H | Cl |
| 069 | Cl | 2-(1,1,1-trifluorobutyl) | H | F |
| 070 | CH₃ | 2-(1,1,1-trifluorobutyl) | H | F |
| 071 | Cl | 2-(1,1,1-trifluorobutyl) | H | Cl |
| 072 | CH₃ | 2-(1,1,1-trifluorobutyl) | H | Cl |
| 073 | Cl | iso-propyl | H | F |
| 074 | CH₃ | iso-propyl | H | F |
| 075 | Cl | iso-propyl | H | Cl |
| 076 | CH₃ | iso-propyl | H | Cl |
| 077 | Cl | sec-butyl | H | F |
| 078 | CH₃ | sec-butyl | H | F |
| 079 | Cl | sec-butyl | H | Cl |
| 080 | CH₃ | sec-butyl | H | Cl |
| 081 | Cl | iso-butyl | H | F |
| 082 | CH₃ | iso-butyl | H | F |
| 083 | Cl | iso-butyl | H | Cl |
| 084 | CH₃ | iso-butyl | H | Cl |
| 085 | Cl | 1,2-dimethylpropyl | H | F |
| 086 | CH₃ | 1,2-dimethylpropyl | H | F |
| 087 | Cl | 1,2-dimethylpropyl | H | Cl |
| 088 | CH₃ | 1,2-dimethylpropyl | H | Cl |
| 089 | Cl | 1,2,2-trimethylpropyl | H | F |
| 090 | CH₃ | 1,2,2-trimethylpropyl | H | F |
| 091 | Cl | 1,2,2-trimethylpropyl | H | Cl |
| 092 | CH₃ | 1,2,2-trimethylpropyl | H | Cl |
| 093 | Cl | cyclopentyl | H | F |
| 094 | CH₃ | cyclopentyl | H | F |
| 095 | Cl | cyclopentyl | H | Cl |
| 096 | CH₃ | cyclopentyl | H | Cl |
| 097 | Cl | cyclohexyl | H | F |
| 098 | CH₃ | cyclohexyl | H | F |
| 099 | Cl | cyclohexyl | H | Cl |
| 100 | CH₃ | cyclohexyl | H | Cl |

| | | | |
|---|---|---|---|
| 101 | Br | —(CH₂)₂—CH(CH₃)—(CH₂)₂— | Cl |

where
a) 101 compounds of formula (I.a):

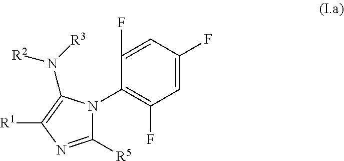

wherein R¹, R², R³ and R⁵ are as defined in Table 1.
b) 101 compounds of formula (I.b):

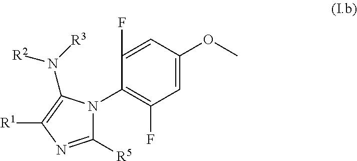

wherein R¹, R², R³ and R⁵ are as defined in Table 1.
c) 101 compounds of formula (I.c):

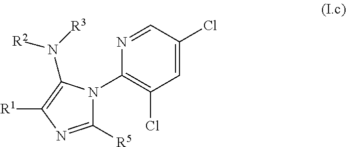

wherein R¹, R², R³ and R⁵ are as defined in Table 1.

Throughout this description, temperatures are given in degrees Celsius, “m.p.” means melting point, “NMR” means nuclear magnetic resonance spectrum; and “%” is percent by weight, unless corresponding concentrations are indicated in other units.

The following abbreviations are used throughout this description:

m.p.=melting point br=broad

s=singlet dd=doublet of doublets

d=doublet dt=doublet of triplets

t=triplet q=quartet

m=multiplet ppm=parts per million

Table 2 shows selected NMR data, all with CDCl₃ as the solvent (unless otherwise stated, no attempt is made to list all characterising data in all cases) for compounds of Table 1.

TABLE 2
Melting point and selected NMR data for compounds of Table 1

| Compound Number | ¹H-NMR data (ppm/number of H's/multiplicity) |
|---|---|
| I.a.003 | 6.87 ppm, 2H, t, J = 7.7 Hz; 3.03 ppm, 2H, dt, |
| | J = 1.9, 11.7 Hz; 2.86 ppm, 2H, bd, J = 11.5 Hz; |
| | 1.53 ppm, 2H, bd, J = 12.4 Hz; 1.48-1.32 ppm, 1H, |
| | m; 0.92-0.82 ppm, 2H, m; 0.86 ppm, 3H, d, |
| | J = 6.5 Hz. |
| I.a.004 | 6.85 ppm, 2H, t, J = 7.85 Hz; 2.9-2.81 ppm, 4H, m; |
| | 2.22 ppm, 3H, s; 1.49 ppm, 2H, bd, J = 12.4 Hz; |
| | 1.35-1.25 ppm, 1H, m; 0.92-0.83 ppm, 2H, m; |
| | 0.85 ppm, 3H, d, J = 6.6 Hz |
| I.b.004 | 6.59 ppm, 2H, d, J = 8.9 Hz; 3.87 ppm, 3H, s; |
| | 2.90 ppm, 2H, bd, J = 11.2 Hz; 2.82 ppm, 2H, dt, |
| | J = 2.3, 11.5 Hz; 2.21 ppm, 3H, s: 1.49 ppm, 2H, |
| | bd, J = 12.2 Hz; 1.35-1.25 ppm, 1H, m; 0.95- |
| | 0.88 ppm, 2H, m; 0.85 ppm, 3H, d, J = 6.6 Hz |
| I.b.101 | 6.61 ppm, 2H, d, J = 9.1 Hz; 3.88 ppm, 3H, s; |
| | 3.02 ppm, 2H, t, J = 11.7 Hz; 2.88 ppm, 2H, bd, |
| | J = 11.5 Hz; 1.51 ppm, 2H, bd, J = 12.8 Hz; 1.37- |
| | 1.27 ppm, 1H, m; 0.94-0.84 ppm, 2H, m; |
| | 0.85 ppm, 3H, d, J = 6.6 Hz |

The compounds according to the present invention can be prepared according to the above-mentioned reaction schemes, in which, unless otherwise stated, the definition of each variable is as defined above for a compound of formula (I).

### BIOLOGICAL EXAMPLES

### Alternaria solani/Tomato/Leaf Disk Preventative (Early Blight)

Tomato leaf disks cv. Baby were placed on water agar in multiwell plates (24-well format) and sprayed with test solutions. Two days after application, the leaf disks were inoculated with a spore suspension of the fungus. After an incubation period at 23° C./21° C. (day/night) and 80% r.h. under a light regime of 12/12 h (light/dark) in a climate cabinet, the activity of a compound was assessed as percent disease control compared to untreated when an appropriate level of disease damage appears in untreated checks (5-7 days after application).

Compounds I.a.004 and I.b.101 at 200 ppm gave at least 80% disease control in this test when compared to untreated control leaf disks under the same conditions, which showed extensive disease development.

Blumeria graminis f. sp. hordei (Erysiphe graminis f. sp. hordei)/Barley/Leaf Disk Preventative (Powdery Mildew on Barley)

Barley leaf segments cv. Hasso were placed on agar in a multiwell plate (24-well format) and sprayed with test solutions. One day after application, the leaf disks was inoculated by shaking powdery mildew infected plants above the test plates. After an incubation period at 20° C. and 60% r.h. under a light regime of 24 h darkness followed by 12 h light/12 h darkness in a climate chamber, the activity of a compound was assessed as percent disease control compared to untreated when an appropriate level of disease damage appeared in untreated checks (5-7 days after application).

Compounds I.a.004, I.b.004 and I.b.101 at 200 ppm gave at least 80% disease control in this test when compared to untreated control leaf disks under the same conditions, which showed extensive disease development.

Puccinia recondita f. sp. tritici/Wheat/Leaf Disk Preventative (Brown Rust)

Wheat leaf segments cv. Kanzler were placed on agar in multiwell plates (24-well format) and sprayed with test solutions. One day after application, the leaf disks were inoculated with a spore suspension of the fungus. After an incubation period at 19° C. and 75% r.h. under a light regime of 12 h light/12 h darkness in a climate cabinet, the activity of a compound was assessed as percent disease control compared to untreated when an appropriate level of disease damage appeared in untreated checks (7-9 days after application).

Compounds I.a.004, I.b.004 and I.b.101 at 200 ppm gave at least 80% disease control in this test when compared to untreated control leaf disks under the same conditions, which showed extensive disease development.

Pyrenophora teres/Barley/Leaf Disk Preventative (Net Blotch)

Barley leaf segments cv. Hasso were placed on agar in a multiwell plate (24-well format) and sprayed with test solutions. Two days after application, the leaf disks were inoculated with a spore suspension of the fungus. After an incubation period at 20° C. and 65% r.h. under a light regime of 12 h light/12 h darkness in a climate cabinet, the activity of a compound was assessed as disease control compared to untreated when an appropriate level of disease damage appears in untreated checks (5-7 days after application).

Compounds I.a.004, I.b.004 and I.b.101 at 200 ppm gave at least 80% disease control in this test when compared to untreated control leaf disks under the same conditions, which showed extensive disease development.

1. A compound of formula I 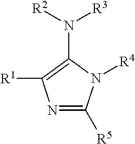
wherein
R¹ is halogen or C₁-C₆alkyl;
R² is an optionally substituted C₁-C₈alkyl, C₂-C₆alkenyl, C₂-C₆alkynyl, C₁-C₇haloalkyl, C₃-C₉cycloalkyl, C₁-C₁₁alkylcycloalkyl or C₁-C₈heterocyclyl;
R³ is hydrogen or C₁-C₇alkyl; or
R² and R³ together with the nitrogen atom to which they are attached form an optionally substituted heterocylic ring;
R⁴ is an optionally substituted aryl or heteroaryl; and
R⁵ is halogen;
said optional substituents comprising one or more substituents independently selected from halogen, alkyl, haloalkyl, cycloalkyl, cycloalkylalkyl, alkenyl, haloalkenyl, cycloalkenyl, alkynyl, haloalkynyl, alkyloxy, haloalkyloxy, cycloalkoxy, alkenyloxy, haloalkenyloxy, alkynyloxy, haloalkenyloxy, alkylthio, haloalkylthio, cycloalkylthio, alkenylthio, alkynylthio, alkylcarbonyl, haloalkylcarbonyl, cycloalkylcarbonyl, alkenylcarbonyl, alkynylcarbonyl, alkoxyalkyl, cyano, nitro, hydroxy, mercapto, amino, alkylamino and dialkylamino;
or an agrochemically usable salt form thereof.

2. A compound according to claim 1 wherein R¹ is fluoro, chloro, bromo, iodo or C₁-C₅alkyl.

3. A compound according to claim 1 wherein R² is an optionally substituted C₁-C₇alkyl, C₂-C₅alkenyl, C₂-C₅alkynyl, C₁-C₆haloalkyl, C₃-C₈cycloalkyl, C₁-C₁₀alkylcycloalkyl or C₁-C₇heterocyclyl.

4. A compound according to claim 1 wherein R³ is hydrogen or C₁-C₆alkyl.

5. A compound according to claim 1 wherein R² and R³ together with the nitrogen atom to which they are attached form an optionally substituted mono or bicylic three- to ten-membered heterocyclic ring, wherein one or two ring carbon atoms can be replaced by oxygen, sulphur or nitrogen.

6. A compound according to claim 1 wherein R⁴ is an optionally substituted phenyl, naphthyl, thienyl, pyridyl, quinolyl or isoquinolyl.

7. A compound according to claim 1 wherein R⁵ is fluoro, chloro, bromo or iodo.

8. A compound according to claim 1 wherein
R¹ is fluoro, chloro, bromo or C₁-C₃alkyl;
R² is an optionally substituted ethyl, iso-propyl, sec-butyl, iso-butyl, 1,2-dimethylpropyl, 1,2,2-trimethylpropyl, 2,2,2-trifluoroethyl, 2-(1,1,1-trifluoropropyl), 2-(1,1,1-trifluorobutyl), cyclopentyl or cyclohexyl;
R³ is hydrogen or C₁-C₄alkyl; or
R² and R³ together with the nitrogen atom to which they are attached form an optionally substituted mono three- to seven-membered heterocyclic ring, wherein one ring carbon atom can be replaced by oxygen or sulphur;
R⁴ is 2,4,6-trifluorophenyl, 2,6-difluoro-4-methoxyphenyl or 3,5-dichloro-pyridin-2-yl; and
R⁵ is fluoro or chloro.

9. A compound according to claim 8 wherein
R¹ is chloro or methyl;
R² is iso-butyl or 2-(1,1,1-trifluoropropyl);
R³ is hydrogen; or
R² and R³ together with the nitrogen atom to which they are attached form a 4-methyl piperidine ring;
R⁴ is 2,4,6-trifluorophenyl or 2,6-difluoro-4-methoxyphenyl; and
R⁵ is fluoro or chloro.

10. A compound according to claim 1 selected from
1-[2,5-Dichloro-3-(2,4,6-trifluoro-phenyl)-3H-imidazol-4-yl]-4-methyl-piperidine;
1-[2-Chloro-3-(2,6-difluoro-4-methoxy-phenyl)-5-methyl-3H-imidazol-4-yl]-4-methyl-piperidine;
1-[5-Chloro-2-fluoro-3-(2,4,6-trifluoro-phenyl)-3H-imidazol-4-yl]-4-methyl-piperidine;
1-[2-Chloro-5-methyl-3-(2,4,6-trifluoro-phenyl)-3H-imidazol-4-yl]-4-methyl-piperidine;
1-[2,5-Dichloro-3-(2,6-difluoro-4-methoxy-phenyl)-3H-imidazol-4-yl]-4-methyl-piperidine;
[2-Chloro-3-(2,6-difluoro-4-methoxy-phenyl)-5-methyl-3H-imidazol-4-yl]-(2,2,2-trifluoro-1-methyl-ethyl)-amine;
[2-Chloro-3-(2,6-difluoro-4-methoxy-phenyl)-5-methyl-3H-imidazol-4-yl]-isobutyl-amine;
1-[2-Chloro-5-methyl-3-(2,4,6-trifluoro-phenyl)-3H-imidazol-4-yl]-4-methyl-piperidine;
1-[2-Chloro-3-(2,6-difluoro-4-methoxy-phenyl)-5-methyl-3H-imidazol-4-yl]-4-methyl-piperidine;
[2-Chloro-5-methyl-3-(2,4,6-trifluoro-phenyl)-3H-imidazol-4-yl]-(2,2,2-trifluoro-1-methyl-ethyl)-amine;
[2-Chloro-3-(2,6-difluoro-4-methoxy-phenyl)-5-methyl-3H-imidazol-4-yl]-(2,2,2-trifluoro-1-methyl-ethyl)-amine;
sec-Butyl-[2-chloro-5-methyl-3-(2,4,6-trifluoro-phenyl)-3H-imidazol-4-yl]-amine;
sec-Butyl-[2-chloro-3-(2,6-difluoro-4-methoxy-phenyl)-5-methyl-3H-imidazol-4-yl]-amine;
[2-Chloro-5-methyl-3-(2,4,6-trifluoro-phenyl)-3H-imidazol-4-yl]-isobutyl-amine;
[2-Chloro-3-(2,6-difluoro-4-methoxy-phenyl)-5-methyl-3H-imidazol-4-yl]-isobutyl-amine;
[2-Chloro-5-methyl-3-(2,4,6-trifluoro-phenyl)-3H-imidazol-4-yl]-(1,2-dimethyl-propyl)-amine;
[2-Chloro-3-(2,6-difluoro-4-methoxy-phenyl)-5-methyl-3H-imidazol-4-yl]-(1,2-dimethyl-propyl)-amine;
4-[2-Chloro-5-methyl-3-(2,4,6-trifluoro-phenyl)-3H-imidazol-4-yl]-morpholine; and
4-[2-Chloro-3-(2,6-difluoro-4-methoxy-phenyl)-5-methyl-3H-imidazol-4-yl]-morpholine.

11. A process for the preparation of a compound of formula I.1, 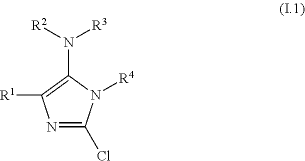
wherein R², R³ and R⁴ are as defined for compound of formula I, and R¹ is halogen or C₁-C₄alkyl, which comprises reacting a compound of formula II, 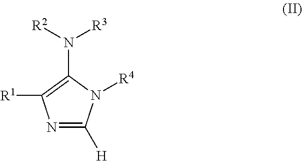
wherein R², R³ and R⁴ are as defined for compound of formula I, and R¹ is halogen or C₁-C₄alkyl, with N-chlorosuccinimide or molecular chlorine.

12. A process for the preparation of a compound of formula I, 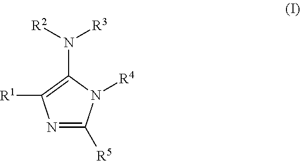
wherein R², R³, R⁴ and R⁵ are as defined for compound of formula I, and R¹ is halogen, which comprises reacting a compound of formula III, 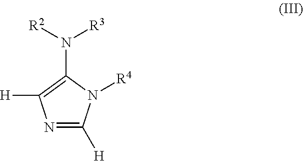
wherein R², R³ and R⁴ are as defined for compound of formula I, with at least 2 equivalents of N-chlorosuccinimide, N-bromosuccinimide or N-iodosuccinimide.

13. A process for the preparation of a compound of formula XV, 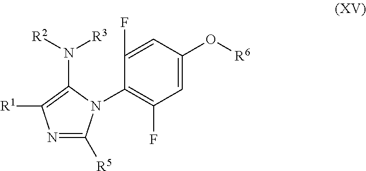
wherein R² and R³ are as defined for compound of formula I, or R² and R³ together with the interjacent nitrogen atom is an optionally substituted heterocyclic ring, R⁵ is alkylthio, R⁶ is C₁-C₄alkyl and R¹ is hydrogen, halogen or C₁-C₄alkyl, which comprises reacting a compound of formula XVI, 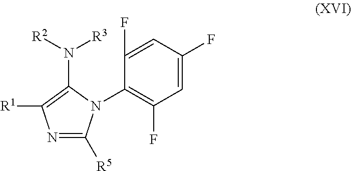
wherein R² and R³ are as defined for compound of formula I, or R² and R³ together with the interjacent nitrogen atom is an optionally substituted heterocyclic ring, R⁵ is alkylthio and R¹ is hydrogen, halogen or C₁-C₄alkyl, with an alkoxide of formula LiOR⁶, NaOR⁶, KOR⁶ wherein R⁶ is C₁-C₄alkyl.

14. A fungicidal composition comprising at least one compound of formula I, as defined in claim 1, in free form or in agrochemically usable salt form, and at least one adjuvant.

15. A composition according to claim 14, which comprises at least one additional fungicidally active compound, preferably selected from the group consisting of azoles, pyrimidinyl carbinoles, 2-amino-pyrimidines, morpholines, anilinopyrimidines, pyrroles, phenylamides, benzimidazoles, dicarboximides, carboxamides, strobilurines, dithiocarbamates, N-halomethylthiotetrahydrophthalimides, copper-compounds, nitrophenols, organo-phosphorus-derivatives, triazolopyrimidines, carboxamides or benzamides.

16. The use of a compound of formula I, as defined in claim 1, for controlling or preventing infestation of plants, harvested food crops, seeds or non-living materials by phytopathogenic microorganisms.

17. A method of controlling or preventing an infestation of crop plants, harvested food crops or non-living materials by phytopathogenic or spoilage microorganisms or organisms potentially harmful to man, which comprises the application of a compound of formula I, as defined in claim 1, to the plant, to parts of the plants or to the locus thereof, to seeds or to any part of the non-living materials.

18. The method according to claim 17 wherein the phytopathogenic microorganisms are fungal organisms.
